(19) United States
(12) Patent Application Publication
(10) Pub. No.: US 2014/0309183 A1
(43) Pub. Date: Oct. 16, 2014

(54) Low-Dose Combination Chemotherapy

(71) Applicant: David Kerr, Deddington (GB)

(72) Inventor: David Kerr, Deddington (GB)

(21) Appl. No.: 14/240,660

(22) PCT Filed: Aug. 24, 2011
(86) PCT No.: PCT/US11/48965

§ 371 Date: May 27, 2014

(51) Int. Cl. A61K 31/7068; A61K 45/06; A61K 31/675; A61K 31/704; A61K 31/506; A61K 31/282

(52) U.S. Cl. CPC A61K 31/7068; A61K 31/506; A61K 31/282; A61K 31/675; A61K 31/704; A61K 45/06
USPC 514/34; 514/49

(57) ABSTRACT

Disclosed herein are compositions comprising at least two compounds in a therapeutically effective composition for treating cancer. Compositions include a single oral dosage form comprising at least two anti-cancer compounds. Also disclosed herein are methods of making and using such compositions.

### BACKGROUND

According to the World Health Organization (WHO), cancer is a leading cause of death worldwide, and accounted for nearly 13% of all deaths in 2008. Deaths from cancer worldwide are expected to continue to rise until 2030, by WHO projections.

Cancer begins with a change in a single cell. The change may be either external or intrinsic. For example, it is known that exposure of a cell to a carcinogen such as certain viruses, certain chemicals, or radiation, leads to DNA alteration that inactivates a “suppressive” gene or activates an “oncogene”. Suppressive genes are growth regulatory genes, which upon mutation, can no longer control cell growth. Oncogenes are initially normal genes (called protoncogenes) that by mutation or altered context of expression become transforming genes. The products of transforming genes cause inappropriate cell growth. More than twenty different normal cellular genes can become oncogenes by genetic alteration. Transformed cells differ from normal cells in many ways, including cell morphology, cell-to-cell interactions, membrane content, cytoskeletal structure, protein secretion, gene expression and mortality (transformed cells can grow indefinitely).

A tumor is an unregulated, disorganized proliferation of cell growth. A tumor is malignant, or cancerous, if it has the properties of invasiveness and metastasis. Invasiveness refers to the tendency of a tumor to enter surrounding tissue, breaking through the basal laminas that define the boundaries of the tissues, thereby often entering the body's circulatory system. Metastasis refers to the tendency of a tumor to migrate to other areas of the body and establish areas of proliferation away from the site of initial appearance.

All of the various cell types of the body can be transformed into benign or malignant tumor cells. The most frequent tumor site is lung, followed by colorectal, breast, prostate, bladder, pancreas, and then ovary. Other prevalent types of cancer include leukemia, central nervous system cancers, including brain cancer, melanoma, lymphoma, erythroleukemia, uterine cancer, and head and neck cancer.

The WHO proposes that almost a third of all cancer deaths may be prevented. Cancer is currently primarily treated with one or a combination of three types of therapies: surgery, radiation, and chemotherapy. Surgery involves the bulk removal of diseased tissue. While surgery is sometimes effective in removing tumors located at certain sites, for example, in the breast, colon, and skin, it cannot be used in the treatment of tumors located in other areas, such as the backbone, nor in the treatment of disseminated neoplastic conditions such as leukemia.

Chemotherapy involves the disruption of cell replication or cell metabolism. It is used in the treatment of leukemia and lymphomas, as well as the majority of solid malignancies, e.g., breast, lung, colorectal, prostate, gynecological, and testicular cancer. Although a number of chemotherapeutic agents have been identified and are currently used for the treatment of cancer, new agents are sought that are efficacious and which exhibit low toxicity toward healthy cells.

### DETAILED DESCRIPTION OF THE INVENTION

The process of discovering and/or developing new chemotherapeutic agents is expensive, time-consuming, and seldom successful. Many prospective new chemotherapeutic agents that show promise in the laboratory fail for one of many reasons in the clinical or regulatory settings. Some agents provide no better efficacy than currently-used agents. Other agents may be effective, but have unacceptable toxicity levels or profiles.

In contrast to developing or discovering new chemotherapeutic agents, new methods of using known chemotherapeutic agents is disclosed herein to provide an alternative route to chemotherapeutic compositions and methods of treatment. The disclosed embodiments encompass chemotherapeutic agents now known, as well as those agents yet to be discovered.

In an embodiment, a composition is provided for treatment of cancer, wherein the composition comprises at least two chemotherapeutic agents, each present in an amount below its respective maximum tolerated dose. In an embodiment, each agent is present in an amount that does not induce greater than the United States National Institutes of Health (NIH) Common Toxicity Criteria Manual (CTC) Grade ½ toxicity. In another embodiment, a method is provided for treatment of cancer, wherein a composition comprising at least two chemotherapeutic agents is administered to a patient having a cancer, further wherein the dosage is adjusted such that each agent is administered in an amount below its respective maximum tolerated dose. In an embodiment, each agent is administered in an amount that does not induce greater than the United States National Institutes of Health (NIH) Common Toxicity Criteria Manual (CTC) Grade ½ toxicity.

In an aspect, disclosed herein are compositions and methods directed to a combination therapeutic for the treatment of cancer in a subject in need thereof, wherein the composition is administered in a single oral therapeutic composition. The preparation and use of such compositions, among others, are described in greater detail elsewhere herein. By way of a non-limiting example, capecitabine and cyclophosphamide form the basis of a single oral therapeutic composition, with additional therapeutic and chemotherapeutic agents added to the composition as needed (e.g., for treating a specific type of cancer, for ameliorating a specific side effect, etc . . . ). By administering the compounds disclosed herein with other chemotherapeutic agents, the therapeutic effect of these compounds can be potentiated. In an embodiment, combination therapies as described herein exert a synergistic effect in treating cancer because each component of the combination acts on a different aspect of the cancer. In another embodiment, the use of such combinations may reduce the dosage of a given conventional chemotherapeutic agent which would be required for a desired therapeutic or prophylactic effect as compared to when that agent is administered as a monotherapy. In an embodiment, these combinations may reduce or eliminate the side effects of conventional single chemotherapy while not interfering with the chemotherapeutic activity of the agents. In an embodiment, such combinations reduce the potential for resistance to single agent therapies, while minimizing any associated toxicity. In another embodiment, such combinations may also increase the efficacy of the conventional agent without increasing the associated toxicity.

### Therapeutic Indications

The compositions and methods disclosed herein are useful for treating cancer, as well as conditions associated with cancer and conditions associated with the treatment of cancer. The term “neoplasia” or “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Representative cancers include, for example, breast, stomach, colon, rectal, liver, pancreatic, lung, cervix uteri, corpus uteri ovary, prostate, testis, bladder, renal, brain/CNS, head and neck, throat, Hodgkin's disease, non-Hodgkin's lymphoma, multiple myeloma, leukemia, melanoma, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, esophagus, larynx, kidney cancer, glioblastoma, hepatocellular, and lymphoma, among others, which may be treated by one or more compounds encompassed by the present disclosure.

The term “tumor” is used to describe a malignant or benign growth.

The term “hyperproliferative disease state” refers to a disease state in which cells are growing in an uncontrolled manner, whether that growth is cancerous or not.

The term “patient” or “subject”, as used herein, describes an animal, encompassing mammals, and further encompassing humans, to whom treatment, including prophylactic treatment, with the compositions according to the present disclosure is provided. For treatment of those conditions or disease states which are specific for a specific animal such as a human patient, the term patient refers to that specific animal.

### Compounds and Compositions

The term “anti-cancer compound” or “anti-cancer agent” is used to describe any compound (including its derivatives) which may be used to treat cancer. Anticancer compounds for use in the compositions and methods disclosed herein may be co-administered in multiples of two or more for the effect that each of these compounds (or their derivative compounds) have on treating cancer in a patient. Anticancer compounds for use in the compositions and methods disclosed herein may also be co-administered in multiples of two or more for the effect that the combined compounds (or their derivative compounds) collectively have on treating cancer in a patient. In an embodiment, coadministration of two or more compounds results in an additive effect. In an embodiment, co-administration of two or more compounds results in a synergistic anti-cancer effect.

Anti-cancer agents for use as embodied herein include, but are not limited to, agents which are broadly characterized as antimetabolites, anthracyclines, fluoropyrimidines, antifolates, vinca alkaloids, inhibitors of topoisomerase I and II, alkylating agents, platinum-based agents, microtubule inhibitors, and taxanes (e.g., paclitaxel). In an aspect, an anti-cancer agent encompassed herein is any anti-cancer agent that can be formulated for oral administration. Anti-cancer compounds encompassed by the present disclosure include, for example, Aldesleukin; Alemtuzumab; alitretinoin; allopurinol; altretamine; amifostine; anastrozole; arsenic trioxide; Asparaginase; bexarotene capsules; bexarotene gel; bleomycin; busulfan intravenous; busulfan oral; calusterone; capecitabine; carboplatin; carmustine; carmustine; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; diflomotecan; docetaxel; doxorubicin; doxorubicin analogs; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); mechlorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; paclitaxel oral preparations; pamidronate; pegademase; Pegaspargase; Pegfilgrastim; pentostatin; pipobroman; plicamycin; mithramycin; porfimer sodium; procarbazine; quinacrine; Rasburicase; Rituximab; S1; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and combinations thereof, among others.

The term “bioactive agent” includes any biologically active agent, including a prodrug form of the active agent, which can be administered in combination with the compositions disclosed herein, and in the methods disclosed herein. In addition to anti-cancer agents as otherwise described above, bioactive agents may include, for example, anti-angiogenic agents, and agents which are useful for the treatment of hyperproliferative diseases, among others. In an embodiment, anticancer compounds for use in the compositions and methods disclosed herein may be co-administered with additional compounds, including, but not limited to, one or more bioactive agents, for the effect that each of these compounds (or derivatives), or for the effect that the combination of compounds, has on enhancing the effect of treating cancer in a patient.

The term “therapeutically effective amount” is used herein, unless otherwise indicated, to describe an amount of a compound which, in context, is used to produce or effect an intended therapeutic result. In an embodiment, the intended therapeutic result relates to the treatment of a hyperproliferative disease state, a tumor including a carcinogenic tumor or other cancer or the treatment of a precancerous lesion or other cell(s) which express abnormal or foreign proteins or immunogens on a cell surface. In certain aspects, the disclosure herein relates to the enhancement of the anti-cancer effect of another anti-cancer compound. This term subsumes all other effective amount or effective concentration terms which are otherwise described in the present application. With respect to an anticancer effect, that effect may be one or more of inhibiting further growth of tumor or cancer cells, inducing an antiangiogenic effect (e.g., by killing tumor endothelial cells), reducing the likelihood or eliminating metastasis or producing cell death in the tumor or cancer cells, resulting in a shrinkage of the tumor or a reduction in the number of cancer cells or preventing the regrowth of a tumor or cancer after the patient's tumor or cancer is in remission. As indicated, an anti-cancer agent may exhibit an anti-cancer effect alone and/or may enhance the ability of another anticancer agent to exhibit an anti-cancer effect.

In an embodiment, a composition is provided for treatment of cancer, wherein the composition comprises at least two chemotherapeutic agents, wherein at least one agent is present in an amount below its respective maximum tolerated dose. In an embodiment, a composition is provided for treatment of cancer, wherein the composition comprises at least two chemotherapeutic agents, each present in an amount below its respective maximum tolerated dose. In an embodiment, a composition is provided for treatment of cancer, wherein the composition comprises at least two chemotherapeutic agents, each present in an amount such that the overall composition dosage is below the maximum tolerated dose for the overall composition.

The term “coadministration” or “combination therapy” is used to describe a therapy in which at least two compounds are used to treat cancer or another disease state or condition as described herein at the same time. In an embodiment, at least two compounds in effective amounts are used to treat cancer or another disease state or condition as described herein at the same time. In another embodiment, at least two compounds, the combination of which comprises an effective amount, are used to treat cancer or another disease state or condition as described herein at the same time. In an embodiment, the result of treatment with the at least two compounds may be additive of the treatment results obtained using each compound separately, either directly additive, or additive to a degree lesser than the results obtained with the two compounds separately. In an embodiment, the result of treatment with the at least two compounds may be synergistic, to varying degrees. In an embodiment, the result of treatment with the at least two compounds may be less than the treatment results obtained using each compound separately. In an aspect, the result of treatment with a composition encompassed herein is such that, for one compound, the result of treatment is less than that obtained with the compound separately, while the results of treatment with respect to the other compounds in the composition are about the same as the results of treatment obtained separately. In an aspect, the result of treatment for at least two compounds is less than that obtained with the compounds separately, while the other compounds in the composition are about the same as the results of treatment obtained separately. In an aspect, the result of treatment for all compounds in the composition is less than that obtained with the compounds separately.

Although the term coadministration encompasses the administration of two active compounds to the patient at the same time, it is not necessary that the compounds be administered to the patient at the same time, although effective amounts of the individual compounds will be present in the patient at the same time.

In an embodiment, a composition is provided for treatment of cancer, wherein the composition comprises at least two chemotherapeutic agents, wherein at least one agent is present in an amount that does not induce greater than Grade ½ toxicity, according to the United States National Institutes of Health (NIH) Common Toxicity Criteria Manual (CTC). In an embodiment, a composition is provided for treatment of cancer, wherein the composition comprises at least two chemotherapeutic agents, each present in an amount that does not induce greater than Grade ½ toxicity, according to the NIH CTC Manual. In an embodiment, a composition is provided for treatment of cancer, wherein the composition comprises at least two chemotherapeutic agents, each present in an amount such that the overall composition dosage does not induce greater than Grade ½ toxicity, according to the NIH CTC Manual.

In an embodiment, a composition encompassed herein has anti-angiogenic activity. In an embodiment, a composition encompassed herein comprises at least one anti-angiogenic agent. In an aspect, one or more chemotherapeutic agents in a composition may have anti-angiogenic activity. In an embodiment, a composition encompassed herein further comprises at least one anti-angiogenic agent which is separate from the chemotherapeutic agents in the composition. In an aspect, a separate anti-angiogenic agent may also have chemotherapeutic activity.

Anti-angiogenic agents include, but are not limited to, a tyrosine kinase inhibitor, a VEGF tyrosine kinase inhibitor, a monoclonal antibody specific for VEGF, angiostatin, endostatin, vasostatin, prolactin, thalidomide, carboxyamidotriazole, prothrombin, interferon-alpha, interferon-beta, interferon-gamma. In an embodiment, an anti-angiogenic agent is an orally-administrable agent. In an embodiment, an anti-angiogenic agent is pazopanib or sorafenib.

The term “pharmaceutically acceptable”, as used herein with respect to a compound or composition, refers to a form of the compound or composition that can increase or enhance the solubility or availability of the compound in a subject, in order to promote or enhance the bioavailability of the compound or composition. In an aspect, the disclosure herein also encompasses pharmaceutically acceptable, hydrates, solvates, stereoisomers, or amorphous solids of the compounds and compositions embodied herein. For example, the term “pharmaceutically acceptable salt” is to describe a salt form of one or more of the compositions herein which are presented to increase the solubility of the compound, for example, in the gastric juices of the patient's gastrointestinal tract in order to promote dissolution and the bioavailability of the compounds and/or compositions. In an embodiment, pharmaceutically acceptable salts include those derived from pharmaceutically acceptable inorganic or organic bases and acids. Suitable salts include those derived from alkali metals such as potassium and sodium, alkaline earth metals such as calcium, magnesium and ammonium salts, among numerous other acids well known in the pharmaceutical art. Sodium and potassium salts are particularly preferred as neutralization salts of carboxylic acids and free acid phosphate containing compositions encompassed by the present disclosure. The term “salt” shall mean any salt consistent with the use of the compounds encompassed by the present disclosure. In the case where the compounds are used in pharmaceutical indications, including the treatment of neoplasia, including cancer, the term “salt” shall mean a pharmaceutically acceptable salt, consistent with the use of the compounds as pharmaceutical agents.

The term “pharmaceutically acceptable derivative” or “derivative”, as used herein, describes any pharmaceutically acceptable prodrug form (such as an ester or ether or other prodrug group) which, upon administration to a patient, provides directly or indirectly the present compound or an active metabolite of the present compound.

In an embodiment, a composition encompassed herein comprises one or more anti-emetic agents. In an embodiment, an anti-emetic agent is selected from the group consisting of a dopamine antagonist, a 5-HT3 receptor antagonist, an H1 histamine receptor antagonist, an NK1 receptor antagonist, a benzodiazepine, a cannabinoid, or a steroid. In an embodiment, an anti-emetic is selected from the group consisting of prednisolone, metoclopramide, stemetil, granesitron, lorazepam, tetrahydrocannabinol, cinnarizine, and prochlorperazine.

### Dosages, Forms and Formulations

The compositions include pharmaceutically acceptable salts of the compounds in the composition. The acids which are used to prepare the pharmaceutically acceptable acid addition salts of the aforementioned compounds are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)] salts, among others.

In an embodiment, compositions comprise base addition salts of the present compounds. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (eg., potassium and sodium) and alkaline earth metal cations (e, calcium and magnesium), ammonium or water-soluble amine addition salts such as N-methylglucamine-(meglumine), and the lower alkanolammonium and other base salts of pharmaceutically acceptable organic amines, among others.

As used herein, the term pharmaceutically acceptable salts or complexes refers to salts or complexes (e.g., solvates, polymorphs) that retain the desired biological activity of the parent compound and exhibit minimal, if any, undesired toxicological effects. Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acids, naphthalenedisulfonic acids, and polygalacturonic acid; (b) base addition salts formed with polyvalent metal cations such as zinc, calcium, bismuth, barium, magnesium, aluminum, copper, cobalt, nickel, cadmium, sodium, potassium, and the like, or with an organic cation formed from N,N-dibenzylethylene-diamine, ammonium, or ethylenediamine; or (c) combinations of (a) and (b); e.g., a zinc tannate salt or the like.

Modifications of a compound can affect the solubility, bioavailability and rate of metabolism of the active species, thus providing control over the delivery of the active species. Further, the modifications can affect the anticancer activity of the compound, in some cases increasing the activity over the parent compound. This can easily be assessed by preparing the derivative and testing its anticancer activity according to the methods encompassed herein, or other methods known to those skilled in the art.

In an embodiment, the compositions may be formulated in a conventional manner using one or more pharmaceutically acceptable carriers and may also be administered in controlled-release formulations. In an embodiment, a controlled-release formulation may release one or more specific compounds prior to the release of the remaining compounds of a therapeutic composition. In an aspect, a single oral dosage form is formulated to release one or more selected compounds prior to the remaining compounds in the controlled-release formulation. In an embodiment, a controlled-release formulation may release the composition as a whole, over a specific period of time, which is, in an aspect, a period of time longer than that otherwise occurring in a formulation that is not a controlled-release formulation. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions encompassed herein may be administered orally. In other embodiments, compositions may be administered parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

The pharmaceutical compositions embodied herein may be orally administered in any orally acceptable dosage form including, but not limited to, capsules, tablets, aqueous suspensions or solutions. In the case of tablets for oral use, carriers which are commonly used include lactose and corn starch. In an embodiment, lubricating agents, such as magnesium stearate, are also added. For oral administration in a capsule form, useful diluents include lactose and/or dried corn starch, as two non-limiting examples. When aqueous suspensions are required for oral use, the active ingredient is combined with emulsifying and suspending agents. If desired, certain sweetening, flavoring or coloring agents may also be added.

The pharmaceutical compositions encompassed by the present disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition encompassed by the present disclosure that may be combined with the carrier materials to produce a single dosage form will vary depending upon the host and disease treated, as well as the particular mode of administration. In an embodiment, as described in detail elsewhere herein, a composition of the invention may be administered orally to a patient continuously (e.g., on a daily basis). In an embodiment, dosage and a dosage regimen is optimized so as not to induce greater than CTC Grade ½ toxicity in the patient.

In an embodiment, compound and/or composition dosage is that which results in less than 100% of conventional MTD doses known in the art. In an embodiment, compound and/or composition dosage is that which results in an MTD between about 50 and about 75% of the conventional MTD known in the art for the particular compound and/or composition. In an embodiment, compound and/or composition dosage is that which results in an MTD between about 25% and about 50% of the conventional MTD known in the art for the particular compound and/or composition. In an embodiment, compound and/or composition dosage is that which results in an MTD between about 10% and about 20% of the conventional MTD known in the art for the particular compound and/or composition. In an embodiment, compound and/or composition dosage is that which results in an MTD between about 5% and about 15% of the conventional MTD known in the art for the particular compound and/or composition. In an embodiment, compound and/or composition dosage is that which results in an MTD of about 10% or less of the conventional MTD known in the art for the particular compound and/or composition.

It will be understood, based on the disclosure set forth herein, in view of the skill in the art, that specific dosage for compounds and compositions encompassed herein may be determined empirically through clinical and/or pharmacokinetic experimentation, and that such dosages may be adjusted according to prespecified toxicity criteria. It will also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

In an embodiment, a composition is formulated to independently include between about 0.1 milligram and about 2000 milligrams of each active compound, including anti-cancer compounds, anti-angiogenic compounds, and anti-emetic compounds, among others, as well as bioactive compounds. In an embodiment, a composition is formulated to independently include any value between about 0.1 milligram and about 2000 milligrams of each active compound, including anti-cancer compounds, anti-angiogenic compounds, and anti-emetic compounds, among others, as well as bioactive compounds. In an embodiment, a composition is formulated to independently include any value between about 0.1 milligram and about 2000 milligrams of each active compound per unit dosage form, including anti-cancer compounds, anti-angiogenic compounds, and anti-emetic compounds, among others, as well as bioactive compounds.

In an embodiment, a composition is formulated to include between about 0.1 milligram and about 1500 milligrams of each active compound. In an embodiment, a composition is formulated to include between about 0.1 milligram and about 1000 milligrams of each active compound. In an embodiment, a composition is formulated to include between about 0.1 milligram and about 750 milligrams of each active compound. In an embodiment, a composition is formulated to include between about 0.5 milligrams and about 600 milligrams of each active compound, between about 1 milligram and about 500 milligrams of each active compound, between about 2 milligrams and about 400 milligrams of each active compound, between about 3 milligrams and about 300 milligrams of each active compound, between about 4 milligrams and about 250 milligrams of each active compound, between about 5 milligrams and about 150 milligrams of each active compound, between about 6 milligrams and about 100 milligrams of each active compound, between about 7 milligrams and about 75 milligrams of each active compound, between about 8 milligrams and about 50 milligrams of each active compound, between about 9 milligrams and about 40 milligrams of each active compound, between about 10 milligrams and about 30 milligrams of each active compound.

In an embodiment, a composition is formulated to include about 0.1 milligrams of an active compound, including anti-cancer compounds, anti-angiogenic compounds, and anti-emetic compounds, among others, as well as bioactive compounds. In an embodiment, a composition is formulated to include about 0.5 millgrams of an active compound, about 1.0 milligram of an active compound, about 2 milligrams of an active compound, about 3 milligrams of an active compound, about 4 milligrams of an active compound, about 5 milligrams of an active compound, about 6 milligrams of an active compound, about 7 milligrams of an active compound, about 8 milligrams of an active compound, about 9 milligrams of an active compound, about 10 milligrams of an active compound, about 15 milligrams of an active compound, about 20 milligrams of an active compound, about 25 milligrams of an active compound, about 30 milligrams of an active compound, about 35 milligrams of an active compound, about 40 milligrams of an active compound, about 45 milligrams of an active compound, about 50 milligrams of an active compound, about 75 milligrams of an active compound, about 100 milligrams of an active compound, about 150 milligrams of an active compound, about 200 milligrams of an active compound, about 250 milligrams of an active compound, about 300 milligrams of an active compound, about 400 milligrams of an active compound, about 500 milligrams of an active compound, about 600 milligrams of an active compound, about 700 milligrams of an active compound, about 800 milligrams of an active compound, about 900 milligrams of an active compound, or about 1000 milligrams of an active compound. In another embodiment, a composition is formulated to include a concentration selected from a range of concentrations of an active compound, wherein the range is selected from any two values set forth above.

In an embodiment, a composition is formulated to include about 0.1 milligrams or less of an active compound, including anti-cancer compounds, anti-angiogenic compounds, and anti-emetic compounds, among others, as well as bioactive compounds. In an embodiment, a composition is formulated to include about 0.5 millgrams or less of an active compound, about 1.0 milligram or less of an active compound, about 2 milligrams or less of an active compound, about 3 milligrams or less of an active compound, about 4 milligrams or less of an active compound, about 5 milligrams or less of an active compound, about 6 milligrams or less of an active compound, about 7 milligrams or less of an active compound, about 8 milligrams or less of an active compound, about 9 milligrams or less of an active compound, about 10 milligrams or less of an active compound, about 15 milligrams or less of an active compound, about 20 milligrams or less of an active compound, about 25 milligrams or less of an active compound, about 30 milligrams or less of an active compound, about 35 milligrams or less of an active compound, about 40 milligrams or less of an active compound, about 45 milligrams or less of an active compound, about 50 milligrams or less of an active compound, about 75 milligrams or less of an active compound, about 100 milligrams or less of an active compound, about 150 milligrams or less of an active compound, about 200 milligrams or less of an active compound, about 250 milligrams or less of an active compound, about 300 milligrams or less of an active compound, about 400 milligrams or less of an active compound, about 500 milligrams or less of an active compound, about 600 milligrams or less of an active compound, about 700 milligrams or less of an active compound, about 800 milligrams or less of an active compound, about 900 milligrams or less of an active compound, about 1000 milligrams or less of an active compound, about 1500 milligrams or less of an active compound, or about 2000 milligrams or less of an active compound.

In an embodiment, a composition is formulated to include about 0.1 milligrams or more of an active compound, including anti-cancer compounds, anti-angiogenic compounds, and anti-emetic compounds, among others, as well as bioactive compounds. In an embodiment, a composition is formulated to include about 0.5 milligrams or more of an active compound, about 1.0 milligram or more of an active compound, about 2 milligrams or more of an active compound, about 3 milligrams or more of an active compound, about 4 milligrams or more of an active compound, about 5 milligrams or more of an active compound, about 6 milligrams or more of an active compound, about 7 milligrams or more of an active compound, about 8 milligrams or more of an active compound, about 9 milligrams or more of an active compound, about 10 milligrams or more of an active compound, about 15 milligrams or more of an active compound, about 20 milligrams or more of an active compound, about 25 milligrams or more of an active compound, about 30 milligrams or more of an active compound, about 35 milligrams or more of an active compound, about 40 milligrams or more of an active compound, about 45 milligrams or more of an active compound, about 50 milligrams or more of an active compound, about 75 milligrams or more of an active compound, about 100 milligrams or more of an active compound, about 150 milligrams or more of an active compound, about 200 milligrams or more of an active compound, about 250 milligrams or more of an active compound, about 300 milligrams or more of an active compound, about 400 milligrams or more of an active compound, about 500 milligrams or more of an active compound, about 600 milligrams or more of an active compound, about 700 milligrams or more of an active compound, about 800 milligrams or more of an active compound, about 900 milligrams or more of an active compound, or about 1000 milligrams or more of an active compound.

In an embodiment, a composition for treating cancer comprises a therapeutically effective amount of a composition comprising a fluoropyrimidine, an alkylating agent, and an anti-emetic. In an embodiment, a composition for treating cancer comprises a therapeutically effective amount of a composition comprising capecitabine, cyclophosphamide, and metoclopramide. In an embodiment, a composition for treating cancer comprises a therapeutically effective amount of a composition comprising about 100 mg to about 500 mg capecitabine and about 10 mg to about 50 mg cyclophosphamide. In an embodiment, a composition for treating cancer comprises a therapeutically effective amount of a composition comprising about 100 mg to about 500 mg capecitabine and about 10 mg to about 50 mg cyclophosphamide, additionally comprising an anti-emetic.

In an embodiment, a composition for treating cancer comprises a therapeutically effective amount of at least two cytotoxic compounds. In an embodiment, a composition for treating cancer comprises a therapeutically effective amount of at least three cytotoxic compounds. In an embodiment, composition for treating cancer comprises a therapeutically effective amount of capecitabine and of cyclophosphamide. In an embodiment, composition for treating cancer comprises a therapeutically effective amount of capecitabine, of cyclophosphamide, and of idarubicin. In an embodiment, composition for treating cancer comprises a therapeutically effective amount of capecitabine, of cyclophosphamide, and of satraplatin.

In an embodiment, a composition for treating cancer is an oral dosage form that comprises a therapeutically effective amount of at least two cytotoxic compounds. In an embodiment, a composition for treating cancer comprises a therapeutically effective amount of at least three cytotoxic compounds. In an embodiment, composition for treating cancer is an oral dosage form that comprises a therapeutically effective amount of capecitabine and of cyclophosphamide. In an embodiment, composition for treating cancer is an oral dosage form that comprises a therapeutically effective amount of capecitabine, of cyclophosphamide, and of idarubicin. In an embodiment, composition for treating cancer is an oral dosage form that comprises a therapeutically effective amount of capecitabine, of cyclophosphamide, and of satraplatin.

In an embodiment, a composition for treating cancer is an single oral dosage form comprising about 1.0 milligram of capecitabine, about 2 milligrams of capecitabine, about 3 milligrams of capecitabine, about 4 milligrams of capecitabine, about 5 milligrams of capecitabine, about 6 milligrams of capecitabine, about 7 milligrams of capecitabine, about 8 milligrams of capecitabine, about 9 milligrams of capecitabine, about 10 milligrams of capecitabine, about 15 milligrams of capecitabine, about 20 milligrams of capecitabine, about 25 milligrams of capecitabine, about 30 milligrams of capecitabine, about 35 milligrams of capecitabine, about 40 milligrams of capecitabine, about 45 milligrams of capecitabine, about 50 milligrams of capecitabine, about 75 milligrams of capecitabine, about 100 milligrams of capecitabine, about 150 milligrams of capecitabine, about 200 milligrams of capecitabine, about 250 milligrams of capecitabine, about 300 milligrams of capecitabine, about 400 milligrams of capecitabine, about 500 milligrams of capecitabine, about 600 milligrams of capecitabine, about 700 milligrams of capecitabine, about 800 milligrams of capecitabine, about 900 milligrams of capecitabine, about 1000 milligrams of capecitabine, about 1500 milligrams of capecitabine, or about 2000 milligrams of capecitabine, and about 1.0 milligram of cyclophosphamide, about 2 milligrams of cyclophosphamide, about 3 milligrams of cyclophosphamide, about 4 milligrams of cyclophosphamide, about 5 milligrams of cyclophosphamide, about 6 milligrams of cyclophosphamide, about 7 milligrams of cyclophosphamide, about 8 milligrams of cyclophosphamide, about 9 milligrams of cyclophosphamide, about 10 milligrams of cyclophosphamide, about 15 milligrams of cyclophosphamide, about 20 milligrams of cyclophosphamide, about 25 milligrams of cyclophosphamide, about 30 milligrams of cyclophosphamide, about 35 milligrams of cyclophosphamide, about 40 milligrams of cyclophosphamide, about 45 milligrams of cyclophosphamide, about 50 milligrams of cyclophosphamide, about 75 milligrams of cyclophosphamide, about 100 milligrams of cyclophosphamide, about 150 milligrams of cyclophosphamide, about 200 milligrams of cyclophosphamide, about 250 milligrams of cyclophosphamide, about 300 milligrams of cyclophosphamide, about 400 milligrams of cyclophosphamide, about 500 milligrams of cyclophosphamide, about 600 milligrams of cyclophosphamide, about 700 milligrams of cyclophosphamide, about 800 milligrams of cyclophosphamide, about 900 milligrams of cyclophosphamide, about 1000 milligrams of cyclophosphamide, about 1500 milligrams of cyclophosphamide, or about 2000 milligrams of cyclophosphamide. In another embodiment, a single oral dosage form comprising capecitabine and cyclophosphamide further comprises about 1.0 milligram of idarubicin, about 2 milligrams of idarubicin, about 3 milligrams of idarubicin, about 4 milligrams of idarubicin, about 5 milligrams of idarubicin, about 6 milligrams of idarubicin, about 7 milligrams of idarubicin, about 8 milligrams of idarubicin, about 9 milligrams of idarubicin, about 10 milligrams of idarubicin, about 15 milligrams of idarubicin, about 20 milligrams of idarubicin, about 25 milligrams of idarubicin, about 30 milligrams of idarubicin, about 35 milligrams of idarubicin, about 40 milligrams of idarubicin, about 45 milligrams of idarubicin, about 50 milligrams of idarubicin, about 75 milligrams of idarubicin, about 100 milligrams of idarubicin, about 150 milligrams of idarubicin, about 200 milligrams of idarubicin, about 250 milligrams of idarubicin, about 300 milligrams of idarubicin, about 400 milligrams of idarubicin, about 500 milligrams of idarubicin, about 600 milligrams of idarubicin, about 700 milligrams of idarubicin, about 800 milligrams of idarubicin, about 900 milligrams of idarubicin, about 1000 milligrams of idarubicin, about 1500 milligrams of idarubicin, or about 2000 milligrams of idarubicin. In another embodiment, a single oral dosage form comprising capecitabine and cyclophosphamide further comprises about 1.0 milligram of satraplatin, about 2 milligrams of satraplatin, about 3 milligrams of satraplatin, about 4 milligrams of satraplatin, about 5 milligrams of satraplatin, about 6 milligrams of satraplatin, about 7 milligrams of satraplatin, about 8 milligrams of satraplatin, about 9 milligrams of satraplatin, about 10 milligrams of satraplatin, about 15 milligrams of satraplatin, about 20 milligrams of satraplatin, about 25 milligrams of satraplatin, about 30 milligrams of satraplatin, about 35 milligrams of satraplatin, about 40 milligrams of satraplatin, about 45 milligrams of satraplatin, about 50 milligrams of satraplatin, about 75 milligrams of satraplatin, about 100 milligrams of satraplatin, about 150 milligrams of satraplatin, about 200 milligrams of satraplatin, about 250 milligrams of satraplatin, about 300 milligrams of satraplatin, about 400 milligrams of satraplatin, about 500 milligrams of satraplatin, about 600 milligrams of satraplatin, about 700 milligrams of satraplatin, about 800 milligrams of satraplatin, about 900 milligrams of satraplatin, about 1000 milligrams of satraplatin, about 1500 milligrams of satraplatin, or about 2000 milligrams of satraplatin.

In an embodiment, a composition for treating cancer is an single oral dosage form comprising about 1.0 milligram or less of capecitabine, about 2 milligrams or less of capecitabine, about 3 milligrams or less of capecitabine, about 4 milligrams or less of capecitabine, about 5 milligrams or less of capecitabine, about 6 milligrams or less of capecitabine, about 7 milligrams or less of capecitabine, about 8 milligrams or less of capecitabine, about 9 milligrams or less of capecitabine, about 10 milligrams or less of capecitabine, about 15 milligrams or less of capecitabine, about 20 milligrams or less of capecitabine, about 25 milligrams or less of capecitabine, about 30 milligrams or less of capecitabine, about 35 milligrams or less of capecitabine, about 40 milligrams or less of capecitabine, about 45 milligrams or less of capecitabine, about 50 milligrams or less of capecitabine, about 75 milligrams or less of capecitabine, about 100 milligrams or less of capecitabine, about 150 milligrams or less of capecitabine, about 200 milligrams or less of capecitabine, about 250 milligrams or less of capecitabine, about 300 milligrams or less of capecitabine, about 400 milligrams or less of capecitabine, about 500 milligrams or less of capecitabine, about 600 milligrams or less of capecitabine, about 700 milligrams or less of capecitabine, about 800 milligrams or less of capecitabine, about 900 milligrams or less of capecitabine, about 1000 milligrams or less of capecitabine, about 1500 milligrams or less of capecitabine, or about 2000 milligrams or less of capecitabine, and about 1.0 milligram or less of cyclophosphamide, about 2 milligrams or less of cyclophosphamide, about 3 milligrams or less of cyclophosphamide, about 4 milligrams or less of cyclophosphamide, about 5 milligrams or less of cyclophosphamide, about 6 milligrams or less of cyclophosphamide, about 7 milligrams or less of cyclophosphamide, about 8 milligrams or less of cyclophosphamide, about 9 milligrams or less of cyclophosphamide, about 10 milligrams or less of cyclophosphamide, about 15 milligrams or less of cyclophosphamide, about 20 milligrams or less of cyclophosphamide, about 25 milligrams or less of cyclophosphamide, about 30 milligrams or less of cyclophosphamide, about 35 milligrams or less of cyclophosphamide, about 40 milligrams or less of cyclophosphamide, about 45 milligrams or less of cyclophosphamide, about 50 milligrams or less of cyclophosphamide, about 75 milligrams or less of cyclophosphamide, about 100 milligrams or less of cyclophosphamide, about 150 milligrams or less of cyclophosphamide, about 200 milligrams or less of cyclophosphamide, about 250 milligrams or less of cyclophosphamide, about 300 milligrams or less of cyclophosphamide, about 400 milligrams or less of cyclophosphamide, about 500 milligrams or less of cyclophosphamide, about 600 milligrams or less of cyclophosphamide, about 700 milligrams or less of cyclophosphamide, about 800 milligrams or less of cyclophosphamide, about 900 milligrams or less of cyclophosphamide, about 1000 milligrams or less of cyclophosphamide, about 1500 milligrams or less of cyclophosphamide, or about 2000 milligrams or less of cyclophosphamide. In another embodiment, a single oral dosage form comprising capecitabine and cyclophosphamide further comprises about 1.0 milligram or less of idarubicin, about 2 milligrams or less of idarubicin, about 3 milligrams or less of idarubicin, about 4 milligrams or less of idarubicin, about 5 milligrams or less of idarubicin, about 6 milligrams or less of idarubicin, about 7 milligrams or less of idarubicin, about 8 milligrams or less of idarubicin, about 9 milligrams or less of idarubicin, about 10 milligrams or less of idarubicin, about 15 milligrams or less of idarubicin, about 20 milligrams or less of idarubicin, about 25 milligrams or less of idarubicin, about 30 milligrams or less of idarubicin, about 35 milligrams or less of idarubicin, about 40 milligrams or less of idarubicin, about 45 milligrams or less of idarubicin, about 50 milligrams or less of idarubicin, about 75 milligrams or less of idarubicin, about 100 milligrams or less of idarubicin, about 150 milligrams or less of idarubicin, about 200 milligrams or less of idarubicin, about 250 milligrams or less of idarubicin, about 300 milligrams or less of idarubicin, about 400 milligrams or less of idarubicin, about 500 milligrams or less of idarubicin, about 600 milligrams or less of idarubicin, about 700 milligrams or less of idarubicin, about 800 milligrams or less of idarubicin, about 900 milligrams or less of idarubicin, about 1000 milligrams or less of idarubicin, about 1500 milligrams or less of idarubicin, or about 2000 milligrams or less of idarubicin. In another embodiment, a single oral dosage form comprising capecitabine and cyclophosphamide further comprises about 1.0 milligram or less of satraplatin, about 2 milligrams or less of satraplatin, about 3 milligrams or less of satraplatin, about 4 milligrams or less of satraplatin, about 5 milligrams or less of satraplatin, about 6 milligrams or less of satraplatin, about 7 milligrams or less of satraplatin, about 8 milligrams or less of satraplatin, about 9 milligrams or less of satraplatin, about 10 milligrams or less of satraplatin, about 15 milligrams or less of satraplatin, about 20 milligrams or less of satraplatin, about 25 milligrams or less of satraplatin, about 30 milligrams or less of satraplatin, about 35 milligrams or less of satraplatin, about 40 milligrams or less of satraplatin, about 45 milligrams or less of satraplatin, about 50 milligrams or less of satraplatin, about 75 milligrams or less of satraplatin, about 100 milligrams or less of satraplatin, about 150 milligrams or less of satraplatin, about 200 milligrams or less of satraplatin, about 250 milligrams or less of satraplatin, about 300 milligrams or less of satraplatin, about 400 milligrams or less of satraplatin, about 500 milligrams or less of satraplatin, about 600 milligrams or less of satraplatin, about 700 milligrams or less of satraplatin, about 800 milligrams or less of satraplatin, about 900 milligrams or less of satraplatin, about 1000 milligrams or less of satraplatin, about 1500 milligrams or less of satraplatin, or about 2000 milligrams or less of satraplatin.

Oral compositions will generally include an inert diluent or an edible carrier. They may be enclosed in gelatin capsules or compressed into tablets. For the purpose of oral therapeutic administration, the active compounds (or prodrugs or derivatives) can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents. These and other agents included in a therapeutic composition may be included from 0.00010% and 99.99% by weight of the final therapeutic composition, including any value within that range, when measured against other components of the therapeutic composition.

The active compound or pharmaceutically acceptable salt thereof, and a composition comprising the same, can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as other anticancer agents, anti-angiogenics, anti-emetics, antibiotics, antifungals, anti-inflammatories, or antiviral compounds, among others.

In an embodiment, the active compounds, or multi-component compositions, are prepared with carriers that will protect the compound or composition against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compounds are then introduced into the container. The container is then swirled by hand to free lipid material from the sides of the container and to disperse lipid aggregates, thereby forming the liposomal suspension.

In an embodiment, a therapeutic composition is prepared by optimizing one or more compounds for use in a dosage form different than that which is typically used for the compound. In an embodiment, a compound that is not typically administered in an oral dosage form is developed for use in an oral dosage form. The chemical and biological assays required for such development are known to one of skill in the art. The disclosure herein provides the skilled artisan with the guidance as to how to prepare such compounds and compositions comprising such compounds.

A wide variety of biological assays have been used and are accepted by those skilled in the art to assess anti-cancer activity of compounds. Any of these methods can be used to evaluate the activity of the compounds and/or formulations disclosed herein. One common method of assessing activity is through the use of the United States National Cancer Institute's (“NCI”) test panels of cancer cell lines. These tests evaluate the in vitro anti-cancer activity of particular compounds, and provide predictive data with respect to the use of tested compounds in vivo. Non-limiting examples of other assays include in vivo evaluations of the effect of a compound on human or mouse tumor cells implanted into or grafted onto nude mice. In an aspect, the effect and efficacy of a compound or combination of compounds can be evaluated in clinical trials on human volunteer patients. Treatment of cancer and side effects of the therapy, among other results, are monitored and used to refine or optimize the compositions and/or methods of treatment using such compositions.

### Methods of Use and Preparation

Compounds and compositions encompassed herein, or derivatives of the same, can be prepared according to the methods encompassed herein and/or known the art.

In an embodiment, the compositions encompassed herein are administered by way of a low-dosage, continuous (i.e., “metronomic”) treatment of a patient having cancer. In an embodiment, the compositions are administered orally. In an embodiment, the compositions are administered in a single dosage form. While not wishing to be bound by any particular theory, a continuous, low-dosage oral administration of a single dosage form chemotherapeutic composition is advantageous for a number of reasons, including prolonged exposure to chemotherapeutic agents, reduced toxicity and side-effect profile for patients, increased compliance with the chemotherapeutic regimen, and potential auxiliary benefits from such a regimen (e.g., anti-angiogenic effects), among others.

In another embodiment, the compositions are administered in multiple dosage forms. In an aspect, different agents are administered in different dosage forms.

In an embodiment, “low-dosage” refers to a dosage of compound that is lower than the MTD of the compound. A more detailed presentation of “low-dosage” is provided elsewhere herein. In an aspect, metronomic therapy using the present compositions has an anti-angiogenic effect on the patient being treated as such. Kerbel et al. (Nature Reviews: Cancer (2004) 423-428; incorporated herein by reference in its entirety) describes preclinical data supporting the concept of low-dose continuous chemotherapy and provides examples illustrating that varying combinations of cyclophosphamide, taxol, doxorubicin, cisplatin, carboplatin, etoposide, vinblastine and fluorouracil are active against a wide range of human tunour xenografts. This anti-tumor activity is enhanced in these models when combined with an anti-angiogenic agent, suggesting that an additional target of the chemotherapy is tumor-associated endothelia.

In an embodiment, a method for treating cancer comprises administering to a mammal in need thereof a therapeutically effective amount of a composition comprising at least two anti-cancer agents, or pharmaceutically acceptable salts, hydrates, solvates, stereoisomers, or amorphous solids thereof, wherein the amount of each anti-cancer agent administered is below the MTD for each agent, the frequency of administration is greater than that used for administration of the MTD of each agent, and the administration of the compound is carried out continuously until there is evidence of treatment of the cancer. In an embodiment, slowing or ceasing tumor progression is a sign of treatment of cancer (e.g., in advanced and/or metastatic cancer). In an embodiment, in an adjuvant setting, an indication of treatment of cancer is found when, for up to and about 2 years of metronomic treatment, there is no return or spread of cancer following apparently curative ablation of the primary tumor (e.g., any combination of surgery, radiotherapy and chemotherapy). In another embodiment, treatment for longer periods of time may be used.

In an embodiment, a method of treating a patient includes administration of a composition encompassed herein to treat cancer, and the treatment of the cancer includes at least one of preventing or slowing the growth of the cancer, preventing the spread of a tumor associated with the cancer, preventing the spread of one or more metastases associated with the cancer, reducing the size of a tumor associated with the cancer, and preventing the recurrence of cancer which was treated previously.

A subject suffering from cancer can be treated by administering to the subject an effective amount of a composition disclosed herein, or derivatives of the same, including pharmaceutically acceptable salts, solvates or polymorphs, thereof optionally in a pharmaceutically acceptable carrier or diluent, either alone, or in combination with other known anticancer or pharmaceutical agents. This treatment can also be administered in conjunction with other conventional cancer therapies, such as radiation treatment or surgery.

The compositions encompassed herein can be administered by any appropriate route, for example, orally, parenterally, intravenously, intradermally, subcutaneously, or topically, in liquid, cream, gel, or solid form, or by aerosol form. In an embodiment, a composition is administered orally. By way of a non-limiting example, a composition comprising capecitabine and cyclophosphamide is administered to a patient orally, in a suitable, single oral dosage form, such that the patient receives a daily dose of about 100 mg to about 500 mg capecitabine and about 10 mg to about 50 mg cyclophosphamide. By way of another non-limiting example, a composition comprising capecitabine, cyclophosphamide and at least one additional compound is administered to a patient orally, in a suitable, single oral dosage form, such that the patient receives a daily dose of about 100 mg to about 500 mg capecitabine and about 10 mg to about 50 mg cyclophosphamide, as well as a therapeutically-effective dose of the at least one additional compound. In an embodiment, the additional compound is another chemotherapeutic compound. In an embodiment, the additional compound is an anti-angiogenic compound. In an embodiment, the additional compound is an anti-emetic. In an embodiment, at least two additional compounds are administered to a patient by way of the single oral dosage form.

In an embodiment, an active compound, and the therapeutic composition as encompassed herein, is included in a pharmaceutically acceptable carrier or diluent in an amount sufficient to deliver to a patient a therapeutically effective amount for the desired indication, without causing serious toxic effects in the patient treated. In an embodiment, a dose of the active compound for all of the herein-mentioned conditions is in the range from about 10 ng/kg to about 300 mg per kilogram body weight of the recipient/patient per day, from about 0.1 mg/kg to about 100 mg/kg per day, from about 0.5 mg/kg to about 25 mg/kg per day. A typical topical dosage will range from 0.01% to 3% wt/wt in a suitable carrier. The compound is conveniently administered in any suitable unit dosage form.

In an embodiment, a therapeutic composition is administered on a schedule once a day. In an embodiment, a therapeutic composition is administered twice a day. In an embodiment, a therapeutic composition is administered three times a day, four times a day, five times a day, or more. In an embodiment, a therapeutic composition is administered less frequently than once a day. In an embodiment, a therapeutic composition is administered once every two days, once every three days, once every four days, once every five days, once every six days, or once every seven days. In an embodiment, a therapeutic composition is administered less frequently than once a week. In an embodiment, a therapeutic composition is administered once a month. In an embodiment, a therapeutic composition is administered twice a month.

In an embodiment, the compound and/or the therapeutic composition, is administered to achieve peak plasma concentrations of the active compound of about 0.00001 mM to about 30 mM, or about 0.1 μM to about 30 μM, with respect to each active compound of an administered composition. This may be achieved, for example, by oral administration of a single dosage form of active ingredients. In another embodiment, it may be achieved by the intravenous injection of a solution or formulation of the active ingredient, optionally in saline, or an aqueous medium or administered as a bolus of the active ingredient.

In an embodiment, the compound and/or the therapeutic composition, is administered in a composition comprising about 100 mg to about 500 mg capecitabine and about 10 mg to about 50 mg cyclophosphamide. In an embodiment, an administered composition comprises about 100 mg to about 500 mg capecitabine and about 10 mg to about 50 mg cyclophosphamide, additionally comprising an anti-emetic. In an embodiment, a method of administering the compound, and the therapeutic composition, comprises providing the subject with a dose of about 100 mg to about 500 mg capecitabine and about 10 mg to about 50 mg cyclophosphamide. In an embodiment, a method of administering the compound, and the therapeutic composition, comprises providing the subject with a daily dose of about 100 mg to about 500 mg capecitabine and about 10 mg to about 50 mg cyclophosphamide. In an aspect, the dosage form comprises flat doses of each compound included in the therapeutic composition. In an embodiment, the dosage form comprises a flat dose of both capecitabine and cyclophosphamide. In an embodiment, the dosage form comprises a flat dose of capecitabine, between about 100 mg and about 500 mg, and a flat dose of cyclophosphamide, between about 10 mg and 50 mg. In an embodiment, the dosage form is oral.

In an embodiment, a method of administering the compound and/or the therapeutic composition comprises providing the subject with a daily dose of about 100 mg to about 500 mg capecitabine and about 10 mg to about 50 mg cyclophosphamide, plus at least one additional cytotoxic compound. In an embodiment, the at least one additional cytotoxic compound is idarubicin. In an embodiment, idarubicin is included in the dosage form so as to administer about 1 mg to about 5 mg idarubicin to a subject daily for three days per month. In an aspect, the subject has breast cancer. In an embodiment, the at least one additional cytotoxic compound is satraplatin. In an embodiment, satraplatin is included in the dosage form so as to administer about 5 mg to about 10 mg satraplatin to a subject daily for fourteen days per month. In an aspect, the subject has at least one of colon cancer, cervical cancer, ovarian cancer, testicular cancer, and lung cancer.

In an aspect, based on the disclosure set forth herein, the skilled artisan will understand how to modify the compounds included in the composition, how to add or remove specific compounds from the composition, and how to adjust the dosage amounts, the dosage frequency, and the route of administration in order to optimize the treatment for a specific subject having a specific type of cancer or cancers. The concentration of active compounds in the composition will depend on absorption, distribution, inactivation, and excretion rates of the compound, as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the individual administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

In an embodiment, the methods and compositions embodied herein are used as a primary or first-line therapy. In an embodiment, the methods and compositions embodied herein are used as a primary or first-line therapy and are not used as maintenance or adjuvant therapy. In an embodiment, the methods and compositions embodied herein are used as a primary or first-line therapy and are also used as maintenance or adjuvant therapy. In an embodiment, the methods and compositions embodied herein are used as maintenance or adjuvant therapy. In an embodiment, the methods and compositions embodied herein are used as maintenance or adjuvant therapy, but are not used as a primary or first-line therapy. By way of a non-limiting example, in an embodiment, in the adjuvant setting, in which a patient has completed conventional treatment with surgical resection of cancer, according to internationally accepted guidelines (eg, National Comprehensive Cancer Network guidelines), the patient is then treated according to the methods and compositions set forth herein. In an aspect, the treatment according to the methods and compositions set forth herein is maintained for about at least one month, about at least two months, about at least three months, about at least six months, about at least nine months, about at least 12 months, about at least 19 months, about at least 24 months, about at least 30 months, or about at least 36\ months. By way of another non-limiting example, in an embodiment, in the adjuvant setting, in which a patient has completed conventional treatment with surgical resection of cancer, followed by local radiotherapy or 6 months chemotherapy, according to internationally accepted guidelines (eg, National Comprehensive Cancer Network guidelines), the patient is then treated according to the methods and compositions set forth herein.

Embodiments of the present disclosure are further described by the following examples. It should be recognized that variations based on the inventive features are within the skill of the ordinary artisan, and that the scope of the disclosure herein should not be limited by the examples. To properly determine the scope of the present disclosure, an interested party should consider the claims herein, and any equivalent thereof. All patents, patent applications, and references cited herein are hereby incorporated by reference in their entirety.

### EXAMPLES

### Example 1

### Clinical Trial for Oral Low-Dose Therapy

The efficacy of treatment of human patients with low-dosage, single-dosage oral combination therapy is determined for capecitabine, oxaliplatin, and pazopanib, according to methods and compositions described herein. An oral dosage, low-dosage form comprising capecitabine, oxaliplatin, and pazopanib is developed such that all components of the oral dosage form are compatible, stable, and biologically active as necessary, within the acceptable parameters required for administration of the dosage form and treatment of the patient.

Patients are monitored for signs of treatment of the cancer, and for signs of adverse events. The course of treatment and dosage levels of the composition, and of individual compounds, may be adjusted as necessary, based on the observed results and effects on the patient, in order to optimize the treatment of cancer in the patient, or in future patients.

### Example 2

### Preparation of Oral Therapeutic Compositions

A therapeutic composition is prepared comprising one or more compounds that are typically administered in an oral dosage form. Once a stable, active, low-dose, single-dosage form composition is prepared, it can be tested in any manner known in the art. In one aspect, the stable, active, low-dose, single-dosage form composition is tested according to the protocol set forth in Example 1. The outcome of the testing and analysis of the composition can be used to optimize and/or refine the dosage form and the overall composition.

### Example 3

### Preparation of Oral Therapeutic Compositions

A therapeutic composition is prepared comprising one or more compounds that are not typically administered in an oral dosage form. For example, an oral composition comprising cyclophosphamide and doxorubicin, as pegylated liposomal doxorubicin, can be prepared. Once a stable, active, low-dose, single-dosage form composition is prepared, it can be tested in any manner known in the art. In one aspect, the stable, active, low-dose, single-dosage form composition is tested according to the protocol set forth in Example 1. The outcome of the testing and analysis of the composition can be used to optimize and/or refine the dosage form and the overall composition.

1. A composition for treating cancer, the composition comprising a therapeutically effective amount of at least two anti-cancer agents, or pharmaceutically acceptable salts, hydrates, solvates, stereoisomers, or amorphous solids thereof, wherein the amount of each anti-cancer agent is below the maximum tolerated dose (MTD) for each agent, further wherein the cancer is susceptible to treatment by the composition.

2. The composition of claim 1, wherein the toxicity profile for the composition is lower than the toxicity profile obtained for any of the anti-cancer agents when used at the MTD.

3. The composition of claim 1, wherein the amount of each anti-cancer agent in the composition is from one-tenth to one-twentieth of the MTD for the anti-cancer agent.

4. The composition of claim 1, wherein the composition further comprises at least one anti-angiogenic agent.

5. The composition of claim 4, wherein the anti-angiogenic agent is selected from the group consisting of pazopanib and sorafenib.

6. The composition of claim 4, wherein the anti-angiogenic agent is an orally-administrable agent.

7. The composition of claim 1, wherein the composition further comprises at least one anti-emetic agent.

8. The composition of claim 7, wherein the anti-emetic agent is selected from the group consisting of a dopamine antagonist, a 5-HT3 receptor antagonist, an H1 histamine receptor antagonist, an NK1 receptor antagonist, a benzodiazepine, a cannabinoid, and a steroid.

9. The composition of claim 7, wherein the anti-emetic is selected from the group consisting of prednisolone, metoclopramide, stemetil, granesitron, lorazepam, tetrahydrocannabinol, cinnarizine, and prochlorperazine.

10. The composition of claim 1, wherein the anti-cancer agents are independently selected from the group consisting of anthracyclines, fluoropyrimidines, antifolates, alkylating agents, vinca alkaloids, taxanes, topoisomerase 1 inhibitors and platinum agents.

11. The composition of claim 1, wherein the anti-cancer agents are independently selected from the group consisting of doxorubicin, a doxorubicin analog, an oral preparation of paclitaxel, methotrexate, cyclophosphamide, capecitabine, S1, 5-fluorouracil, vinerolbine, cisplatin, oxaliplatin, irinotecan, diflomotecan, and etoposide.

12. The composition of claim 11, further comprising at least one anti-angiogenic agent.

13. The composition of claim 11, further comprising at least one anti-emetic agent.

14. The composition of claim 12, further comprising at least one anti-emetic agent.

15. A composition for treating cancer comprising a therapeutically effective amount of a composition comprising a fluoropyrimidine, an alkylating agent, and an anti-emetic.

16. The composition of claim 15, wherein fluoropyrimidine is present at about 500 mg, an alkylating agent is present at about 25 mg, and an anti-emetic is present at about 2.5 mg to about 10 mg.

17. The composition of claim 16, wherein the fluoropyrimidine is capecitabine, the alkylating agent is cyclophosphamide, and the anti-emetic is metoclopramide.

18. A pharmaceutical composition for treating cancer, the composition comprising:
(a) a therapeutically effective amount of the composition of claim 1; and
(b) one or more pharmaceutically acceptable carriers, diluents, and excipients therefor.

19. A method for treating cancer comprising administering to a mammal in need thereof a therapeutically effective amount of a composition comprising at least two anti-cancer agents, or pharmaceutically acceptable salts, hydrates, solvates, stereoisomers, or amorphous solids thereof, wherein:
a. the amount of each anti-cancer agent administered is below the maximum tolerated dose (MTD) for each agent;
b. the frequency of administration is greater than that used for administration of the MTD of each agent; and
c. the administration of the compound is carried out continuously until there is evidence of tumor progression,
further wherein the cancer is susceptible to treatment by the amount of the composition administered.

20. A method for treating cancer comprising administering to a mammal in need thereof a therapeutically effective amount of a composition comprising at least two anti-cancer agents, or pharmaceutically acceptable salts, hydrates, solvates, stereoisomers, or amorphous solids thereof, wherein:
a. the amount of each anti-cancer agent administered is below the maximum tolerated dose (MTD) for each agent;
b. the frequency of administration is greater than that used for administration of the MTD of each agent; and
c. the administration of the compound is carried out continuously for up to two years following apparently curative ablation of the primary tumor,
further wherein the cancer is susceptible to treatment by the amount of the composition administered.

21. The method of claim 19, wherein the composition is administered on a schedule selected from the group consisting of q.d., b.i.d., and t.i.d.

22. The method of claim 19, wherein the composition further comprises at least one anti-angiogenic compound.

23. The method of claim 19, wherein the composition further comprises at least one anti-emetic compound.

24. The method of claim 19, wherein the treatment of the cancer includes at least one of preventing or slowing the growth of the cancer, preventing the spread of a tumor associated with the cancer, preventing the spread of one or more metastases associated with the cancer, reducing the size of a tumor associated with the cancer, and preventing the recurrence of cancer treated previously.

25. The method of claim 19, wherein the cancer is selected from the group consisting of breast cancer, cervical cancer, colorectal cancer, lung cancer, prostate cancer, ovarian cancer, glioblastoma, renal cancer and hepatocellular cancer.

26. The method of claim 25, wherein the cancer is metastatic.

27. A method for treating cancer comprising administering to a mammal in need thereof a therapeutically effective amount of a composition comprising a fluoropyrimidine, an alkylating agent, and an anti-emetic, wherein the composition is administered to the mammal once a day.

28. The method of claim 27, wherein fluoropyrimidine is present at about 500 mg, an alkylating agent is present at about 25 mg, and an anti-emetic is present at about 2.5 mg to about 10 mg.

29. The method of claim 28, wherein the fluoropyrimidine is capecitabine, the alkylating agent is cyclophosphamide, and the anti-emetic is selected from the group consisting of prednisolone and metoclopramide.

30. A method for treating cancer comprising administering to a mammal in need thereof a therapeutically effective amount of at least two anti-cancer agents, or pharmaceutically acceptable salts, hydrates, solvates, stereoisomers, or amorphous solids thereof, wherein the anti-cancer agents are administered simultaneously, wherein the amount of each anti-cancer agent administered is below the maximum tolerated dose (MTD) for each agent, and wherein the cancer is susceptible to treatment by the administration of the anti-cancer agents.

31. The method of claim 30, wherein at least one anti-angiogenic compound is administered simultaneously.

32. The method of claim 30, wherein at least one anti-emetic compound is administered simultaneously.
